(19) United States
(12) Patent Application Publication
(10) Pub. No.: US 2016/0125170 A1
(43) Pub. Date: May 5, 2016

(54) DYNAMIC ANALYSIS OF HEALTH AND MEDICAL DATA APPLIED TO CLINICAL TRIALS

(71) Applicant: Marc Lauren Abramowitz, Palo Alto, CA (US)

(72) Inventor: Marc Lauren Abramowitz, Palo Alto, CA (US)

(21) Appl. No.: 14/926,408

(22) Filed: Oct. 29, 2015

Related U.S. Application Data
(60) Provisional application No. 62/072,368, filed on Oct. 29, 2014.
(60) Provisional application No. 62/086,125, filed on Dec. 1, 2014.

(51) Int. Cl. G06F 19/00; G06F 17/30; G06Q 10/06; H04L 29/06

(52) U.S. Cl. CPC G06F 19/363; H04L 63/08; G06F 17/30867; G06Q 10/06395

(57) ABSTRACT

Methods, devices, systems and computer program products determine suitability of an individual for participation in a drug trial. One method for assessment of suitability of an individual for participation in a drug trial includes receiving a message that provides an identity of an individual and a request for a drug trial suitability assessment for the individual. In response, medical, health or drug-related data is obtained from a plurality of data sources. One or more of the data sources is a real-time data source that is updated on a continual basis. The method further includes filtering the information to produce a customized data set based on the individual's identity and a phase of drug assessment trial. Such customized data set is changeable based on real-time changes in the information obtained from the data sources.

### CROSS REFERENCE TO RELATED APPLICATIONS

This applications claims priority to U.S. Provisional Patent Application No. 62/072,368, filed on Oct. 29, 2014, entitled DYNAMIC MEDICAL DATA ANALYSIS AND RELATED PRODUCTS, and U.S. Provisional Patent Application No. 62/086,125, filed on Dec. 1, 2014, entitled DYNAMIC ANALYSIS OF HEALTH AND MEDICAL DATA, which are hereby incorporated by reference in their entirety.

### TECHNICAL FIELD

The present disclosure relates generally to systems, apparatuses, methods and computer program that are stored on non-transitory storage media (collectively referred to as the “technology”) related to collecting and analyzing medical and health related data, and determining and/or providing assessments that modify and/or enhance clinical trial procedures and other applications.

### BACKGROUND

Clinical trials are prospective biomedical or behavioral research studies on human subjects that are designed to answer specific questions about biomedical or behavioral interventions (novel vaccines, drugs, treatments, functional foods, dietary supplements, devices or new ways of using known interventions), generating safety and efficacy data. They are conducted only after satisfactory information has been gathered that satisfies health authority/ethics committee approval in the country where approval of the therapy is sought. Depending on product type and development stage, investigators initially enroll volunteers and/or patients into small pilot studies, and subsequently conduct progressively larger scale comparative studies. As positive safety and efficacy data are gathered, the number of patients typically increases. Clinical trials can vary in size, and can involve a single research entity in one country or multiple entities in multiple countries.

### SUMMARY OF CERTAIN EMBODIMENTS

The disclosed technology relates to methods, devices, systems and computer program products that enable the determination of suitability of an individual for participation in a clinical or drug assessment trial, and improve drug discovery process by collecting, analyzing and integrating real time data into different aspect of drug discovery process.

Some aspects of the disclosed technology relates to a computer program product that is embodied on one or more computer readable media and includes program code for receiving a first message from a first entity, where the first message comprising an identity of the individual and a request for a drug trial suitability assessment for the individual. The computer program product also includes program code for, in response to the first message, obtaining information comprising medical, health or drug-related data from a plurality of data sources, where one or more of the plurality of data sources is a real-time data source with data that is updated on a continual basis. The computer program product also includes program code for filtering the information obtained from the plurality of data sources to reduce the information comprising the medical, health or drug-related data to produce a customized data set based on at least the identity of the individual and a phase of drug assessment trial, where the customized data set is changeable in response to real-time changes in the information obtained from the plurality of data sources. The computer program product additionally includes program code for using the customized data set to produce a drug trial suitability metric comprising information indicative of the individual's estimated ability to remain in, or benefit from, the drug trial.

One aspect of the disclosed technology relates to a method for assessment of insurance risk that includes receiving a first message from an insurance provider, where the first message includes an identity of an individual and a request for an insurability risk assessment for the individual for a particular type of insurance policy. The method also includes, in response to the first message, obtaining information comprising medical, health or drug-related data from a plurality of data sources, where one or more of the plurality of data sources is a real-time data source with data that is updated on a continual basis. The method further includes filtering the information obtained from the plurality of data sources to reduce the information comprising the medical, health or drug-related data to produce a customized data set based on at least the identity of the individual and the type of insurance policy. The customized data set is changeable in response to real-time changes in the information obtained from the plurality of data sources. The above noted method additional includes using the customized data set to produce an insurability risk metric comprising information indicative of the individual's estimated a health assessment that is relevant to the particular type of insurance policy.

Another aspect of the disclosed embodiments relates to a computer program product that embodied on one or more non-transitory computer readable media and includes computer code for receiving a first message from an insurance provider, where the first message comprising an identity of an individual and a request for an insurability risk assessment for the individual for a particular type of insurance policy. The computer program product also includes computer code for, in response to the first message, obtaining information comprising medical, health or drug-related data from a plurality of data sources, where one or more of the plurality of data sources is a real-time data source with data that is updated on a continual basis. The computer code additionally includes computer code for, filtering the information obtained from the plurality of data sources to reduce the information comprising the medical, health or drug-related data to produce a customized data set based on at least the identity of the individual and the type of insurance policy, where the customized data set is changeable in response to real-time changes in the information obtained from the plurality of data sources, and computer code for, using the customized data set to produce an insurability risk metric comprising information indicative of the individual's estimated a health assessment that is relevant to the particular type of insurance policy.

Another aspect of the disclosed technology relates to a system for assessment of insurance risk that includes a data aggregation and analysis component implemented at least partially using electronic circuits, and including a front end, an identification engine, a customization engine, a filter engine, a decision engine and a non-transitory computer readable storage. The above noted system also includes a plurality of data sources coupled to at least the data aggregation and analysis component. In particular, the front end is coupled to at least a communication link and includes an interface to receive data or information from one or more of: a client device, an insurance provider device, or the plurality of data sources. The identification engine is coupled to at least the front end to receive an identity of an individual and to authenticate the identity, and the customization engine is coupled to the front end to receive information provided by the insurance provider device indicative of a request for an insurability risk assessment for the individual for a particular type of insurance policy. The filter engine is coupled to at least the plurality of data sources an the non-transitory computer readable storage to obtain information comprising medical, health or drug-related data from the plurality of data sources including at least one real-time data source with data that is updated on a continual basis. The filter engine filters the information obtained from the plurality of data sources to reduce the information comprising the medical, health or drug-related data to produce a customized data set based on at least the identity of the individual and the type of insurance policy, where the customized data set is changeable in response to real-time changes in the information obtained from the plurality of data sources. The decision engine is coupled to at least the filter engine to use the customized data set to produce an insurability risk metric comprising information indicative of the individual's estimated a health assessment that is relevant to the particular type of insurance policy.

### BRIEF DESCRIPTION OF THE DRAWINGS

FIG. 1. is a block diagram of a basic and suitable computer that may employ aspects of the described technology.

FIG. 2. is a block diagram illustrating a simple, yet suitable system in which aspects of the described technology may operate in a networked computer environment.

FIG. 3 is an exemplary diagram that shows interactions among an insurance provider, a data aggregation and analysis system, a client, and a data source in accordance with an exemplary embodiment.

FIG. 4 illustrates the connectivity amongst different components of a system in accordance with an exemplary embodiment.

FIG. 5 illustrates various components of a data source and a data aggregation and analysis system in accordance with an exemplary embodiment.

FIG. 6 illustrates a data aggregation and analysis system and the associated interactions among its various components in accordance with an exemplary embodiment

FIG. 7 illustrates a block diagram of a device that can be implemented as part of the disclosed devices and systems.

FIG. 8 illustrates a set of exemplary operations that can be carried out to provide an insurance risk metric in accordance with an exemplary embodiment.

FIG. 9 illustrates a set of exemplary operations that can be used to assess an individual's suitability to participate in, or benefit from, a drug assessment trial in accordance with an exemplary embodiment.

### DETAILED DESCRIPTION

The recent proliferation of computer networks and related technologies has created a vast ocean of information that is produced on a daily, hourly, or sometimes real-time, basis. This information, which is sometimes referred to as Big Data, is all-encompassing and includes the collection of numerous data sets so large and complex that it is difficult to analyze. Somewhere in this large collection of data, important medical and health-related information is buried, which cannot be effectively accessed and/or cannot be properly combined with or correlated with additional data to improve the accuracy and viability of life or health insurance policies, or premiums.

Some aspects of the disclosed technology relate to improving the drug discovery, as well as subsequent analysis, of a drug's efficacy, side effects, and other long and short term issues at the final stages of drug approval process, and even long after the approval process has been completed. Developing a new drug from original idea to the launch of a finished product is a complex process, which can take 12-15 years and cost in excess of $1 billion. It may take many years to select a target for a costly drug discovery program. Once a target has been chosen, the molecules which possess suitable characteristics to make acceptable drugs are identified and further validation is often required prior to progression into the lead discovery phase. During lead discovery, an intensive search ensues to find a drug-like small molecule or biological therapeutic, typically termed a development candidate, that will progress into preclinical, and if successful, into clinical development and ultimately be a marketed medicine.

As described herein, clinical trials are prospective biomedical or behavioral research studies on human subjects that are designed to answer specific questions about biomedical or behavioral interventions (novel vaccines, drugs, treatments, functional foods, dietary supplements, devices or new ways of using known interventions), generating safety and efficacy data. They are conducted only after satisfactory information has been gathered that satisfies health authority/ethics committee approval in the country where approval of the therapy is sought. Depending on product type and development stage, investigators initially enroll volunteers and/or patients into small pilot studies, and subsequently conduct progressively larger scale comparative studies. As positive safety and efficacy data are gathered, the number of patients typically increases. Clinical trials can vary in size, and can involve a single research entity in one country or multiple entities in multiple countries.

Clinical trials involving new drugs are commonly classified into four phases. Phase 0: Pharmacodynamics and Pharmacokinetics. Phase 0 trials are the first-in-human trials. Single subtherapeutic doses of the study drug are often given to a small number of subjects (10 to 15) to gather preliminary data on the agent's pharmacodynamics (what the drug does to the body) and pharmacokinetics (what the body does to the drugs). In Phase 1 trials, researchers test an experimental drug or treatment in a small group of people (20-80) to evaluate its safety, determine a safe dosage range, and identify side effects. In Phase 2 trials, the experimental treatment is given to a larger group of people (100-300) to see if it is effective and to further evaluate its safety. In Phase 3 trials, the treatment is given to large groups of people (1,000-3,000) to confirm its effectiveness, monitor side effects, compare it to commonly used treatments, and collect information that will allow it to be safely used. In Phase 4 trials, post-marketing studies delineate additional information, including the treatment's risks, benefits, and optimal use.

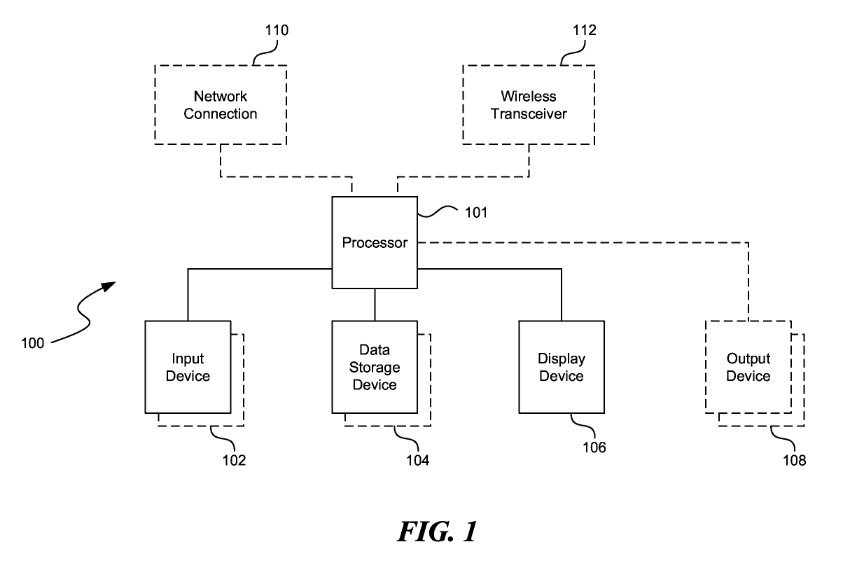

The disclosed embodiments allow the medical and health related information to be used to improve drug discovery and clinical trial (including post-trial) procedures, by enabling, generating, and/or determining an individualized assessment of a person's (trial candidate's) health characteristics and propensities in order to ascertain or determine the person's suitability for participation in clinical trials, the person's prospects for responding to drug treatment at different phases of the clinical trials, including a phase after the completion of the clinical trials. Referring to FIG. 1, an exemplary embodiment of the described technology employs a computer 100, such as a personal computer or workstation, having one or more processors 101 coupled to one or more user input devices 102 and data storage devices 104. The computer 100 can also be coupled to at least one output device such as a display device 106 and one or more optional additional output devices 108 (e.g., printer, plotter, speakers, tactile or olfactory output devices, etc.). The computer 100 may be coupled to external computers, such as via an optional network connection 110, a wireless transceiver 112, or other types of networks.

The input devices 102 may include a keyboard, a pointing device such as a mouse, and described technology for receiving human voice, touch, and/or sight (e.g., a microphone, a touch screen, and/or smart glasses). Other input devices 102 are possible, such as a joystick, pen, game pad, scanner, digital camera, video camera, and the like. The data storage devices 104 may include any type of computer-readable media that can store data accessible by the computer 100, such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to or node on a network, such as a LAN, WAN, or the Internet (not shown in FIG. 1).

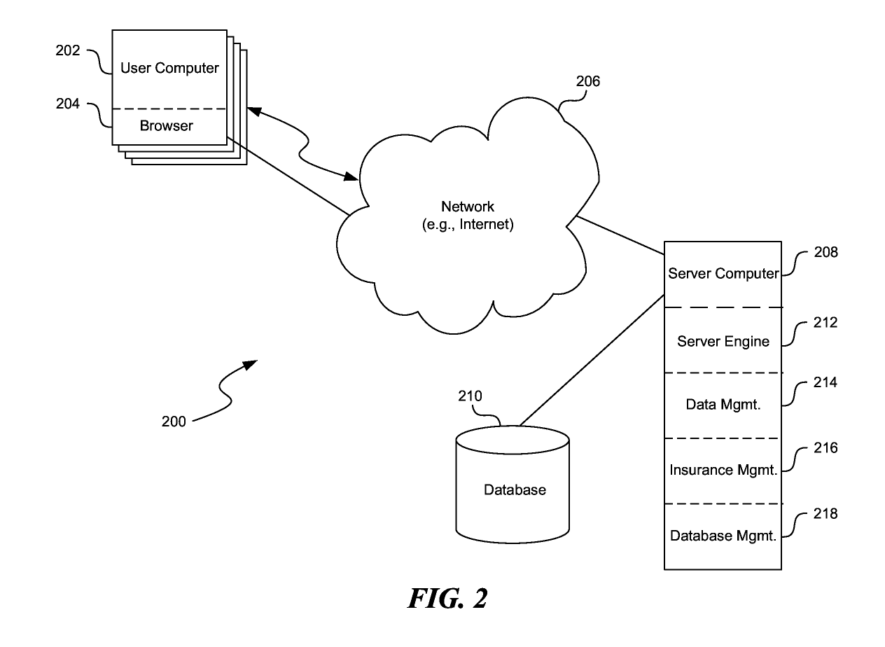

Aspects of the described technology may be practiced in a variety of other computing environments. For example, referring to FIG. 2, a distributed computing environment with a network interface includes one or more user computers 202 (e.g., mobile devices, desktops, servers, etc.) in a system 200, each of which can include a graphical user interface (GUI) program component (e.g., a thin client component) 204 that permits the user computer 202 to access and exchange data, such as network, security data and/or health related data, with a network 206 such as a LAN or the Internet, including web sites, ftp sites, live feeds, and data repositories within a portion of the network 206. The user computers 202 may be substantially similar to the computer described above with respect to FIG. 1. The user computers 202 may be personal computers (PCs) or mobile devices, such as laptops, mobile phones, or tablets. The user computers 202 may connect to the network 206 wirelessly or through the use of a wired connection. Wireless connectivity may include any forms of wireless technology, such as a radio access technology used in wireless LANs or mobile standards such as 2G/3G/4G/LTE. The user computers 202 may include other program components, such as a filter component, an operating system, one or more application programs (e.g., security applications, word processing applications, spreadsheet applications, or Internet-enabled applications), and the like. The user computers 202 may be general-purpose devices that can be programmed to run various types of applications, or they may be single-purpose devices optimized or limited to a particular function or class of functions. More importantly, any application program for providing a graphical user interface to users may be employed, as described in detail below. For example, a mobile application or “app” has been contemplated, such as one used in Apple's® iPhone® or iPad® products, Microsoft® products, Nokia® products, or Android®-based products. In some exemplary configuration of the system 200, the user computers 202 resides at an insurance company, while in another exemplary configuration, the user computers 202 may be located at a health organization.

At least one server computer 208, coupled to the network 206, performs some or all of the functions for receiving, routing, and storing of electronic messages, such as medical data, weather-related data, data related to natural or other disasters, web pages, audio signals, electronic images, and/or other data. While the Internet is shown, a private network, such as an intranet, may be preferred in some applications. The network may have a client-server architecture, in which a computer is dedicated to serving other client computers, or it may have other architectures, such as a peer-to-peer, in which one or more computers serve simultaneously as servers and clients. A database or databases 210, coupled to the server computer(s), store some content (e.g., security-related data, health related data, weather information, etc.) exchanged between the user computers; however, content may be stored in a flat or semi-structured file that is local to or remote of the server computer 208. The server computer(s), including the database(s), may employ security measures to inhibit malicious attacks on the system and to preserve the integrity of the messages and data stored therein (e.g., firewall systems, secure socket layers (SSL), password protection schemes, encryption, and the like).

The server computer 208 may include a server engine 212, a data management component 214, an insurance management component 216, and a database management component 218. The server engine 212 can perform processing and operating system level tasks. The data management component(s) 214 handle creation, streaming, processing and/or routing of medical, health or drug-related data, as well as non-medical data, such as weather, natural or man-made disasters, and the like. Data management components 214, in various embodiments, includes other components and/or technology. Users may access the server computer 208 by means of a network path associated therewith. The insurance management component 216 handles processes and technologies that support the collection, managing, and publishing of insurance-related data and information. The database management component 218 includes storage and retrieval tasks with respect to the database, queries to the database, and storage of data. In some embodiments, multiple server computers 208 each having one or more of the components 212-218 may be utilized. In general, the user computer 202 receives data input by the user and transmits such input data to the server computer 208. The server computer 208 then queries the database 210, retrieves requested pages, performs computations and/or provides output data back to the user computer 202, typically for visual display to the user. Additionally, or alternatively, the user computers 202 may automatically, and/or based on user computers' 202 settings/preferences, receive various information, such as alerts, updates, health/life/long-term care insurance assessments, efficacy information, etc., from the server computer 208.

As described herein, certain aspects of the disclosed technology can be implemented as a system (e.g., a real-time system) that receives medical and health-related information, such as information generated in real-time or near-real-time, from already-existing aggregators, in addition to individual users, and individual organizations. The health related data obtained from the individuals may have originated from personal health monitors, insurance forms, social media websites, and the like. Such a system can then provide insurance risk assessment for consumption by insurance companies, or other organizations that offer insurance products, and/or may perform assessments to identify individuals determined and/or predicted to be suitable for certain clinical trials, such as within one or more stages of a drug trial.

Such a system can provide vastly improved performances that would have been unsatisfactorily conducted in-part by the insurance companies, the operations that would have been performed unsatisfactorily by big data providers, while providing many unique features that cannot be provided by conventional systems. Most of the big medical data that are provided by the existing services can be irrelevant, or be of little relevance, to the tasks of personalized health, life or a long-term care insurance risk assessment. Furthermore, the disclosed technology provides various filters that can reduce large amounts of collected or received data and identify data that relevant to such determinations and/or predictions, even when the collected data includes rapidly changing (e.g., real-time or semi-real-time) health data, environmental factors, personalized habits, and other factors.

Availability of different types of relevant data allows improved insurance risk assessment or drug trial candidates assessment that are based on interactions of those different types of data and factors, which may enable a more accurate personalized insurance profile to be generated and/or the selection or matching of clinical trials to suitable individuals. For example, the disclosed technology can use and integrate different factors, such as health-related outbreaks (e.g., flu, Ebola, etc.), information related to natural disasters (e.g., tornadoes, cold fronts, earthquakes) and personalized health profiles (e.g., age, existing medical conditions, medications, etc.), to create real-time suitability or match assessments based on not only such individual factors, but also the interactions between those factors.

For example, such an assessment can provide information that is personalized for a 35-year old User A in Buffalo, N.Y., who suffers from asthma, in the event of a cold front that is anticipated to last for 3 weeks. If the interactions between the different factors were not take into account (e.g., the anticipated cold front was ignored), a different (and inaccurate) short term insurability and/or trial suitability would have been produced. Thus, the customized suitability assessments can be provided for, and may remain valid for, any time period that may be needed (e.g., daily, weekly, monthly, etc.) and can be based on many factors e.g., drug trial data, impending weather changes, disease outbreaks, (impending) fires, etc.—all obtained via the disclosed real-time system. This way, both long-term and short-term health/life/long-term care insurance risk assessments can take place.

The system can further provide a list of options to an insurance company as to which data/habits/medicines/conditions to track per individual (or group of individuals). In some embodiments, the insurance company can select items of interest, and change those items iteratively as the needs of the insurance company change. In some embodiments, the insured can be notified and provided with recommended actions that are based on the real-time assessments (e.g., get a flu shot before the end of the month to avoid paying a higher premium next month). In other embodiments, the insurance assessments can be used to provide a variable insurance premium (e.g., daily or weekly—rather than the current way of annual assessment/payment).

The system of the present application further includes privacy controls and mechanisms that are compliant with various privacy regulations, such as HIPPA.

Another feature of the disclosed technology is its ability to detect insurance fraud. For example, fraud can be detected based on the computed profile based on the individualized data that is obtained through various sources of data. Such a computed profile gives an indication as to the types/amounts of claims that are expected, which can then be compared to actual claims that are filed in order to detect potential fraud patterns.

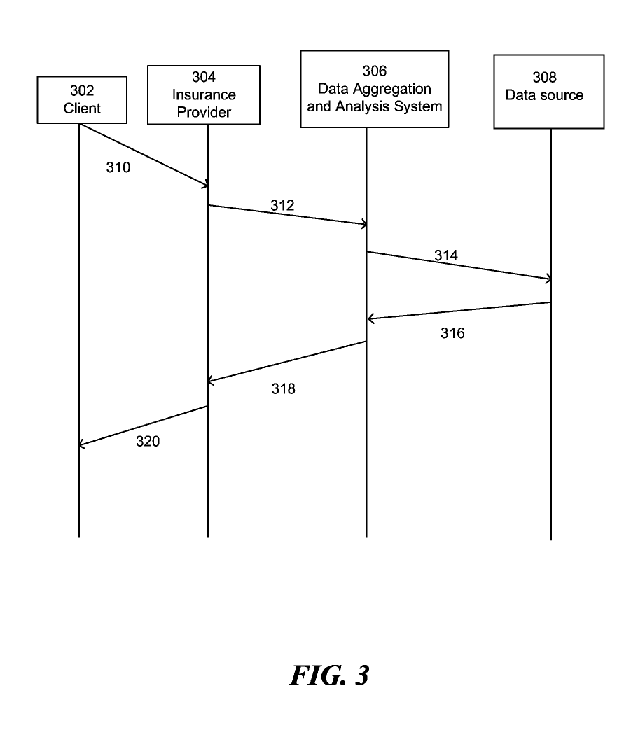

FIG. 3 is an exemplary diagram that shows interactions among an insurance provider 304, a data aggregation and analysis system 306, a client 302, and a data source 308, in accordance with an exemplary embodiment. At 310, a client 302 requests an insurance policy from an insurance provider 304. The client 302 provides some information to the insurance provider 304, as well. The information provided by the client 302 to the insurance provider 304 can include the basic identification information of the client 302. It may also include some information about the client's health history, family history, or other health related information. The information provided by the client 302 to the insurance provider 304 may also include the client's previous insurance history and claim history. The policy requested by the client 302 may be a specific policy designed by the insurance provider 304 for the client 302 specifically. The policy can be a short-term policy, such as a month coverage, or can be a long term policy, such as a health insurance coverage for one year, a life insurance policy or a long-term care insurance policy.

Referring again to FIG. 3, at 312, based on the provided information from the client 302, the insurance provider 304 requests related data from a data aggregation and analysis system 306. The data aggregation and analysis system 306 may store, or have ready access to, the requested information and therefore can provide such information readily to the insurance provider 304. The data aggregation and analysis system 306 uses, at least in-part, the personal identification information provided by the client 302 to find data related to the client 302. As will be further described in the sections that follow, the data aggregation and analysis system 306 can use data provided by other users or organizations, and/or data that is collected by other sources to produce the relevant information for the insurance provider 304.

A 314, the data aggregation and analysis system 306 further collects data from the data source 308, before providing the needed data to the insurance provider 304. The collected data from data source 308 may have information related to the current policy request made on 310 by the client 302 to the insurance provider 304, which were not stored by the data aggregation and analysis system 306. For example, if the insurance policy requested is a new policy or a specific policy designed for the client 302, there is a likelihood that some additional information about the client 302 may be needed. In another example, if the client 302 does not fit into a normal class risk profile for a policy, the data aggregation and analysis system 306 may need additional and refined data, such as recent data that is not stored or collected before, to determine client 302's risk profile.

Referring back to FIG. 3, at 316, the data source 308 provides the requested data to the data aggregation and analysis system 306. The transmission of such data from the data source 308 to the data aggregation and analysis system 306 is through a network, and may take place multiple times, even though only one connection 316 is shown in FIG. 3. The data transferred from the data source 308 to the data aggregation and analysis system 306 may contain images, video, text, or other types of information. In one example, such data is in a pre-defined format, or may be other loosely defined collection of data. At 318, the data aggregation and analysis system 306 provides a decision or feedback to the insurance provider 304, based on the data obtained from the data source 308, the information provided by the insurance provider 304, or the data provided by the client 302. In another example, such decision may be made by the insurance provider 304, and the data aggregation and analysis system 306 may only provide the refined data or feedback that is needed to make such a decision. For instance, the feedback provided at 318 may be processed, filtered, and organized information based on raw data collected at 316.

At 320, the insurance provider 304 provides a result to the client 302. The result provided at 320 may be an approval of the policy requested by the client 302 at 310. The result may also contain a price information on how much it costs to purchase the policy and the duration of the policy. The result at 320 may contain information on what the client can do to qualify for the insurance policy or a discounted insurance policy, such as to provide additional information and documents, to make improvements in diet an exercise habits, make certain number of visits to the primary care provider, and the like.

It should be noted that while the communications between the different entities in FIG. 3 are illustrated using a single, one-directional arrow, in some embodiments, each such communication may include more than one communication (back and forth) between the depicted entities. For example, the insurance provider 304 may request, and receive, additional information from the client 302; the data aggregation and analysis system 306 may request, and receive, additional information from the insurance provider 304, and so on.

In one implementation, the operations performed by the insurance provider 304, the data aggregation and analysis system 306, and the data source 308 are carried out on one computer. In another implementation, the operations performed by the insurance provider 304, the data aggregation and analysis system 306, and the data source 308 are carried out on different computers, systems, or platforms.

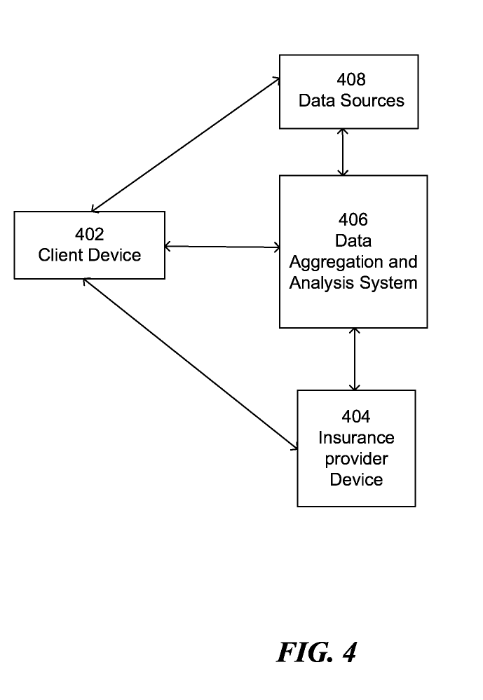

FIG. 4 illustrates the connectivity amongst different components of the system in accordance with an exemplary embodiment. The insurance provider 404 may be a health insurance provider, or a house insurance provider. In another implementation, the insurance provider device 404 provides a combination of insurance policies covering various assets and risks. The insurance provider device 404 can also construct risk models and determine an insurance policy for individuals and groups of individuals. The insurance provider device 404 is coupled to the data aggregation and analysis system 406 to send and receive various information, data and commands, as, for example, illustrated in FIG. 3. The insurance provider device 404 is also coupled to the user device 402 to communicate send and receive various information, including insurance policy requests, personal data, and other information, as, for example, discussed in connection with FIG. 3.

The client device 402 or the insurance provider device 404 may be implemented using a hardware architecture that is described, for example, in connection with FIG. 1. For instance, the client device 402 can be a personal device (e.g., a laptop, a tablet, as smart phone, etc.) of a particular user that allows the provision of personal information to the insurance provider device 404. In another implementation, the client device 402 can be computer system of an organization and can provide the insurance provider device 404 organizational identification information. The insurance policy requested by the client device 402 can be for a short term insurance policy, a long term insurance policy, or both. For instance, the requested insurance information can be for a life insurance policy, a medical insurance policy, a long-term care insurance policy or insurance policies for athletes, doctors, actors, models, etc., which are affected on health of the insured. The insurance policy requested by client 402 may be changeable at a certain time, or it can be a fixed policy that can not be changed during the life time of the policy.

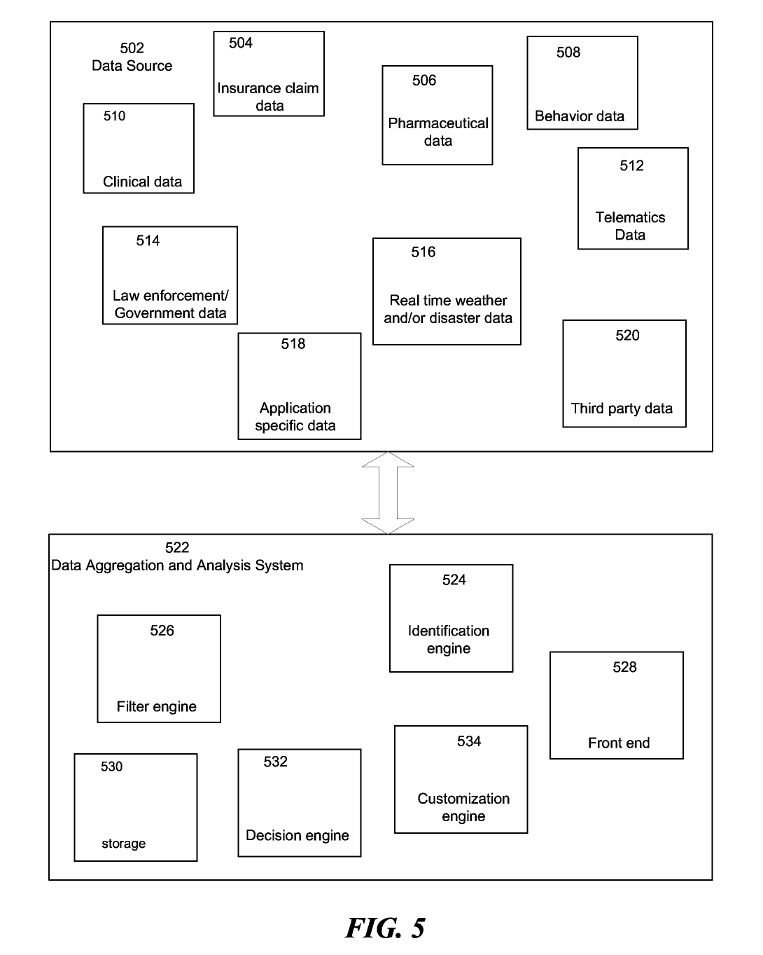

The data source(s) 408, which will be described in further detail in FIG. 5, comprise computer device and/or storage devices that produce, retain, and/or obtain a variety of data, including but not limited to, one or more of clinical data related to drug development, insurance claim data, pharmaceutical R&D data, behavior data, telematics data, real time weather, geographic or disaster data, application specific data, law enforcement, government data, or any third party data. In one implementation, the data source 408 also includes data provided by an individual user, such as a user using the client device 402.

As will be detailed in connection with FIG. 5, in one implementation, the data aggregation and analysis system 406 includes various component such as a front end, an identification engine, a customization engine, a filter engine, a storage, and a decision engine. In one exemplary embodiment, the hardware architecture of the data aggregation and analysis system 406 is similar to those illustrated in FIG. 2 in connection with the computer server 208 and the associated components such as the server engine 212, data management 214 component, insurance management 216 component, and database management component 218.

One set of exemplary interactions among the various components of FIG. 4 were previously described in connection with FIG. 3. It is, however, understood that the interactions among insurance provider device 404, the data aggregation and analysis system 406, the client device 402, and the data source 408, can be more complex than the sequence diagram shown in FIG. 3. For example, the client device 402 may directly interact with the data aggregation and analysis system 406. The data aggregation and analysis system 406 may periodically collect data from the client device 402 directly without going through the insurance provider 404 or the data source 408. In one implementation, the data aggregation and analysis system 406 can design, deploy, or utilize new or existing sensors to track a user's daily activities that are measured by, or collected by, the client device 402. Additional information obtained from the client device 402 can include health history and family history, which, for example, are voluntarily provided by the user when making hospital visits. Such sensor data and additional information can be used to determine the user's health habits and health status, and to accordingly adjust the insurance risk assessment and the associated insurance premium.

As will be clarified further in the sections that follow, the system that is described in FIG. 4 provides many advantages and features by obtaining data from a multitude of data sources, requesting personalized and customized information, providing filtering operations, and iteratively fulfilling the needs of the client device 402 and the insurance provider device 404.

FIG. 5 illustrates various components of a data source 502 and a data aggregation and analysis system 522 in accordance with an exemplary embodiment. It should be noted that FIG. 5 can also be construed as a system that includes a plurality of data sources (e.g., the data sources illustrates as part of the data source 502) and a data aggregation and analysis component (e.g., the data aggregation and analysis system 522).

The components depicted as part of the data source 502 can be implemented using hardware memory devices that can be accessed by a processor to retrieve and/or store information. In some implementations, each of the components of the data source 502 can include many database systems that run on multiple computers or microprocessors. For example, in some embodiments, one or more of the depicted components is a networked server system and/or a cloud system. Such systems can hold publicly accessible information and/or propriety information that are available upon payment of a fee. One of the advantageous of the disclosed technology relates to its ability to aggregate a multitude of free, paid, or restricted-access data sources as part of one system in order to allow individual companies (e.g., client companies) to gain access to a customized set of data that would not be otherwise available (or feasible to obtain) to that client. This way, not only the need for payment of multiple subscription services is avoided, but an insurance company is further assured to receive up-to-date information that is extracted from a multitude of data sources, and at the same time, is narrowly tailored and individualized to fulfill a specific request by a client.

As illustrated in FIG. 5, the data source 502 can include a clinical data source 510, an insurance claim data source 504, a pharmaceutical R&D data source 506, a behavior data source 508, a telematics data source 512, a law enforcement and government data source 514, a real time weather and disaster data source 516, an application specific data source 518, and any third party data source 520. In some implementations, data sources such as the clinical data source 510, the insurance claim data source 504, the pharmaceutical R&D data source 506, and the behavior data source 508, identify a client by his/her name or other identification information (e.g., a social security number, an ID number, etc.). In some instances, the data may be anonymous, but may include sufficient demographic, age, weight, etc. information that allows a reasonably accurate insurance assessment of a client. In some implementations, the data provided by some data sources, such as the real time weather and/or disaster data source 516, may not be explicitly attributed to a client, and other mechanisms, such as the geographic location and its correlation with the client's identification, may be needed to associated the information obtained from the data source to a particular client.

The clinical data source 510 includes patient data stored in a computer-based information system, such as the basic electronic medical records (EMSs) used by physicians and hospitals, the health-information exchanges (HIEs) used by hospitals, or drug trial information obtained as a result of phases 0 through 4 of drug discovery process, as well as additional data associated with long-term effects, efficacy and issues related to particular drugs. In one exemplary implementation, the clinical data source 510 is coupled to, and collects part of the drug trial information, from online communities and social networks such as Facebook, Twitter or other sites, that allow individuals to discuss and share their experiences with a particular drug or therapeutic remedy, including long-term side effects, efficacy or other concerns. Additional patient data may be directly obtained from patients through, for example, personalized health monitors or other devices that are capable of obtaining or measuring patient information and transmitting them to a database. In different geographic locations, the clinical data 510 may be different.

The insurance claim data source 504 can include insurance claims and cost data that describe what services were provided and how they were reimbursed for various policy holders and the amounts of reimbursement. The insurance claim data source 504 can also include data that is collected from many different insurance companies over a period of time and is aggregated to produce a comprehensive database. The pharmaceutical R&D data source 506 includes data that describes drugs therapeutic mechanism of action, target behavior in the body, and side effects and toxicity, as well as drug trial information obtained as a result of phases 0 through 4 of drug discovery process. In one implementation, the pharmaceutical R&D data source 506 includes data collected from many different pharmaceutical R&D companies or service providers, over a period of time. It should be noted that some of the data sources, such as the pharmaceutical R&D data source 506 and the clinical data source 510 may include overlapping or redundant data. One feature of the disclosed technology relates to evaluation of such redundant or overlapping data to filter out the redundant and/or irrelevant information.

The behavior data source 508 includes behavior and sentiment data that describes activities and preferences, both inside and outside the healthcare and insurance context. In one implementation, the behavior data source 508 include data about clients finances, buying preferences, and other characteristics through companies that aggregate and sell consumer information. The behavior data source 508 can further include data collected online from online communities and social networks such as Facebook, LinkedIn, and other sites. The behavior data source 508 can be collected from many companies such as grocery stores, retail stores, banks, credit unions, credit card companies, or other kinds of financial institutions.

The telematics data source 512 includes data generated by telematics methods. Telematics is an interdisciplinary field encompassing telecommunications, vehicular technologies, road transportation, road safety, electrical engineering (sensors, instrumentation, wireless communications, etc.), computer science (multimedia, Internet, etc.). In one implementation, the telematics data is generated by a GPS-enabled tracker that monitors medicine usage by patients. Alternatively, the telematics data can be generated by a mobile application that allows the user to input medical data, or to receive medical data from other monitoring devices.

The real time weather and/or disaster data source 516 can provide data obtained from agencies that monitor or forecast weather patterns or disasters. Such disasters can include natural disasters, such as earthquakes, volcano eruptions, solar flares, etc., and man-made disasters, such as nuclear plant meltdowns, outbreak of a war, oil and natural gas accidents, etc., as well as disease outbreaks, such as Ebola, SARS, Flu, etc. Such data can be used to predict the near or distant future risks of the client and is often associated with a geographic location or region. In one implementation, the data obtained from the real time weather and/or disaster data source 516 is processed in conjunction with additional data, such as the home address of a client, to enable the production of insurance risk assessment for particular clients.

The law enforcement and government data source 514 provides data that can be used to check for fraud history, criminal history, aliases or other names that a client may have used, residence history, and other information. The law enforcement and government data source 514 can thus be used verify the authenticity of the information provided by the clients, or received from other data sources, as well as to uncover any fraudulent or criminal acts that a client may have committed in the past. For example, the law enforcement and government data source 514 can be used to resolve the many affiliated names used by a client. In one implementation, the law enforcement and government data source 514 is collected from many national or international law enforcement agencies such as FBI, CIA, Interpol, or other local, national or international law enforcement agencies, such as police departments of various cities and states.

The third party data source 520 includes data provided by other data aggregators or data providers, which may include raw data, or data that is processed in some way. Such data can be received from existing systems and services, such as Axxiom, Accurint, Optuminsight, ActiveHealth, Healthcore, Transcelerate Biopharma, the Medicare and Medicaid EHR Incentive Programs and others. As noted earlier, such third part data sources 520 often produce large amounts of data that includes duplicative and irrelevant information. The disclosed technology utilizes such third party data sources 520 as one of many sources of data, while providing effective filtering and processing operations that enables the discovery of the proverbial needle in the haystack. To this end, the third party data can be augmented with specific data that is customized to be received by disclosed system, and the collective data sources are processed to produce personalized insurance assessments on a real-time basis.

The application specific data source 518 is generated by the data aggregation and analysis system to fulfill a specific need, such as an insurance need of an insurance provider. For example, the application specific data source 518 can be generated by the data aggregation and analysis system 522 in response to a specific request by an insurance provider. The application specific data source 518 can be updated based on new data received from other data sources, revisions to the requests received from the insurance provider, or both. For instance, in one implementation, the application specific data source 518 be populated with particular clinical data, pharmaceutical R&D data, behavior data, or telematics data that are processed or filtered by the data aggregation and analysis system 522 to conform to the requirements established by a request from the insurance provider.

FIG. 5 further illustrates various component of a data aggregation and analysis system 522 that includes a front end 528, an identification engine 524, a customization engine 534, a filter engine 526, a storage 530, and a decision engine 532. In one implementation, the components that are described as part of data aggregation and analysis system 522 are implemented at least partially in hardware including electronic circuits, such as implementations via an ASIC, FPGA, or a digital signal processor (DSP).

The front end 528 receives input from, and provide output to, other components such as an insurance provider device, a client device or a data source. For example, the front end 528 can directly accept input from a client. In one implementation, the front end 528 contains an interface, such as a GUI, to help the users to input data and display data to the users. The GUI can, for example, be displayed on a web browser running on a computer or a microprocessor. In some implementations, the front end 528 can receive input simultaneously from multiple devices, such as a client device, an insurance provider device, and from one or more data sources.

The identification engine 524 identifies the client. For example, a client may provide one name to the insurance provider, while he/she may have used many other names in the past. In this example, the identification engine 524 uses various data sources such as the law enforcement and government data source 514 to check for different names used by the client. In another example, the identification engine 524 also obtain and verify an email address, social security number, date of birth, locations, residence history, and any other information that can be used to identify the client.

The customization engine 534 is activated in response to an insurance provider's request for a specific type of data that may not currently exist in the data aggregation and analysis system 522. In such a scenario, the data aggregation and analysis system 522 provides a communication mechanism so that the insurance provider device can request a particular customized data to be generated by the data aggregation and analysis system 522. For example, an insurance provider device can request a customized insurance risk assessment for a reporter that makes frequent international trips to Ebola-inflicted countries in West Africa. In this example, the customization engine 534 creates an application specific data source that receives information from the weather and/or disaster data source 516, telematics data source 512 (e.g., that reports body temperature readings of the individual), clinical data source 510 (e.g., that obtains information regarding the latest Ebola drug trial results) or other data sources. The customization engine 534 then utilizes filters (e.g., as part of the customization engine 534 or the filter engine 526) to filter out the relevant information. For example, if the person of interest has only traveled to Liberia, the filter removes information from the weather and/or disaster data source 516 that relates to Ebola outbreak in Siena Leon. Thus, the customization engine 534 can process data provided by an insurance provider, a client or a data source, and produce customized information regarding the insurance policy, or the risk profile. It should be noted that the application specific data source 518 can collect data via connections to the other data sources that are illustrated in FIG. 5, and/or the system can set up a connection to a different data source (not listed) that may be needed to acquire the application specific data.

The filter engine 526 is used to analyze data received from various data sources, such as the ones depicted as part of data source 502. There may be many conflicting data, out of date data, which will be removed by the data filter engine 526. In one implementation, the filter engine 526 organizes the results in a coherent and consistent fashion, such as data that is sorted by time or by relevance. In one implementation, the filter engine 526 organizes the data based on the client identification; the identity of the client may be authenticated or verified by the identification engine 524.

The storage 530 is used to store the filtered data from filter engine 526, so that it can be used for future purposes. The storage 530 can be a memory device (e.g., RAM, ROM, etc.), a hard disk, a flash drive, and so on. The storage 530 can be used to store any data received from the front end 528, or any other components of the data aggregation and analysis system 522, as well as computer program codes that may be retrieved and executed by a processor to perform the various disclosed operations.

The decision engine 532 includes decision logic for computations that lead to a decision based on the filtered data produced by the filter engine 526. In one implementation, the decision engine 532 includes an algorithm that implements a predetermined risk model such as statistics-based model. For example, filter engine 526 can produce several parameters that are considered important in conducting an insurance risk assessment for an individual (e.g., age and body mass index (BMI) of the person, family history of coronary disease, level of daily exercise, type of occupation, proximity to natural Radon-emitting soils, weight changes in the past year, etc.). The risk model can assign particular weights to each of these parameters in coming up with a weighted average insurance risk assessment (e.g., in the range 1 to 100) that is indicative of the likelihood of the person needing medical care (as well as the type and amount of medical care) in the next month, next six months or next year. The data aggregation and analysis system 522 can thus produce an insurability risk metric that includes the weighted average insurance risk assessment (e.g., in the range 1 to 100). In some embodiments, the metric also includes information as to the particular statistics-based model that was used to produce the risk assessments, and any assumptions that may have been made in producing the risk assessments. Typically, such assumptions are made to simplify the model or the computations of the risk assessment (e.g., restricting the geographic area to a particular region, limiting how far back the data must go, etc.). In one embodiment, the insurability risk metric can include several sets of risk assessment data (e.g., based on different models, based on different assumptions, for different types of insurance policies, etc.).

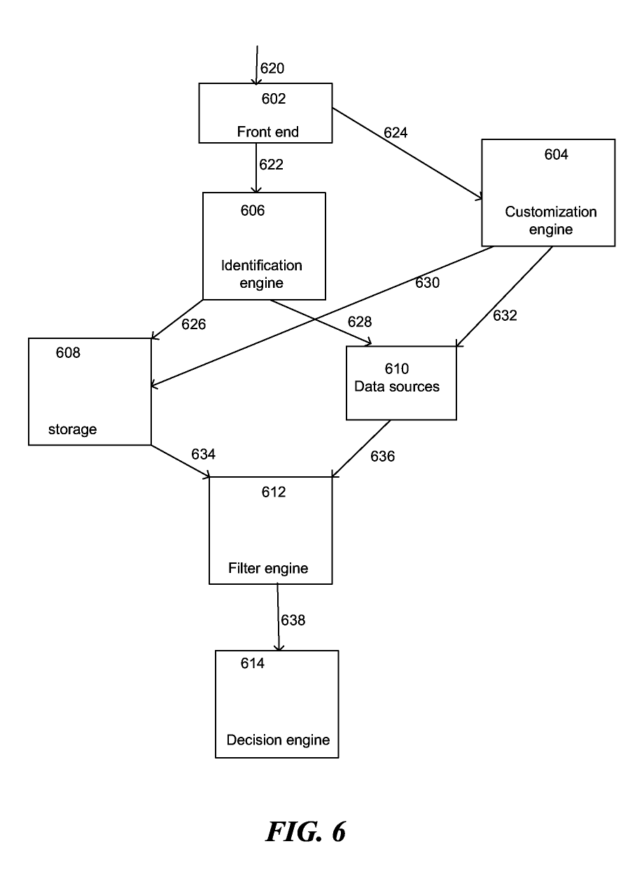

FIG. 6 illustrates a data aggregation and analysis system and the associated interactions among its various components in accordance with an exemplary embodiment. At 620, an input is received at the front end 602. The input may be a request for data from an insurance provider, a data from a data source, or from a client. In one implementation, the input to the front end 602 is accepted through a GUI interface. In some implementations, the input to the front end 602 is accepted from another computer through a computer-to-computer communication link. The front end 622 processes the received data. For example, the processing can include parsing the received data to extract identification information. At 622, at least part of the data processed by the front end 622 that includes one or more forms of identification information is provided to the front end 602. In one implementation, the identification information includes one or more of a name, an email address, a social security number, a date of birth, a current location, a residence history of a client that can be used to identify the client.

The data that is received by the front end 602 can include particular requests. At 604, such requests are provided to the customization engine 604 to generate the new data (e.g., data templates, date sources, etc.) which is not currently established in the data aggregation and analysis system. The customization may be done on the data collected or on the policy requested.

At 626 and 630, the customized request, the client identification information, or the customized data may be sent to the storage 608 to be stored in the data aggregation and analysis system. If the requested data is not in the storage, the data aggregation and analysis system may, at 628, send out a request to the data sources 610 to gather more data.

At 634 and 636, after all the data is gathered from the storage 608 or from data sources 610, the data is passed to the filter engine 612 to be analyzed. In one implementation, there are many conflicting data, out of date data, duplicate data, or irrelevant data which are removed by the data filter engine 612. In one implementation, the filter engine 612 also organizes the results to produce a coherent and consistent data that is sorted in a predetermined order, such as based on time or by relevance. For example, sorting by relevance can produce ordered entries that are sorted based on their relevance to the type of insurance policy requested, or relevance to the individual client. Sorting by time can produce entries that are, for example, listed in the descending order of occurrence, with the most recent data being listed first and the oldest data being listed last. At 638, the filtered and organized data is provided to the decision engine 614 which makes a decision based on the filtered data. As noted earlier, the decision engine 614 can implement a predetermined risk model, such as statistics-based model.

The interactions among the various components shown in FIG. 6 are only for illustration purposes and are not limiting. For example, there may be other additional interactions that are not shown. Furthermore, the communications between different components are shown as one-sided arrows. It is understood, however, that bidirectional communications can take place among the various components.

Another aspect of the disclosed embodiments relates to facilitating assessment of risks associated with long-term care and determination of premiums for long-term care insurance. While the majority of people that require long-term care are over 65 years of age, a sizeable number of younger adults are also in need of long-term care. For example, a study published in 2003 estimated that 36 million Americans under age 65 were in need of long term care. Long term care includes a range of services and benefits that a person may need to be able to carry out his/her daily activities that can persist for many years (e.g., until the end of life). Thus long-term care is not only medical care, but includes assistance with the basic personal tasks of everyday life, such as, bathing, dressing, walking, caring for incontinence, eating and other basic personal hygiene and routine physical activities. Other components of long-term care can include assistance with various tasks that allow a person to maintain a reasonable social life beyond the basic survival needs. These can include, for example, assistance with housework, managing money, shopping for groceries or clothes, using the telephone or other communication devices, caring for pets, responding to emergency alerts such as fire alarms and the like.

Determining the level and duration of such long-term care depends on several factors, which in turn, determine the premium for receiving such services and benefits. These factors include, but are not limited to, the age of the person, the gender of the person (e.g., women typically outlive men), disability of the person, health status of the person (e.g., chronic conditions such as diabetes, high blood pressure), family history, diet, personal habits (e.g., levels of exercise, smoking, drinking), living arrangements (e.g., living alone, in a family), geographical location of residence (e.g., in regions with extreme climates, country of residence), coverage under other insurance policies (e.g., Medicare coverage), and other factors.

The disclosed system can further enable the use of medical, health or drug-related data that is obtained from a plurality of data sources to provide a customized assessment of insurance risks and premiums for such long-term care insurance policies. Such customized information provides a better assessment of the associated risks, and enables projection of the needed benefits that more accurately represent the level and duration of care. For example, while it may be statistically true that, when averaged over a large population sample, females outlive males, such a generalized assumption may be completely irrelevant to a female that, for example, is taking a particular medication that has been recently associated with having certain side effects, or to having undesirable interactions if taken with another medication. In a traditional long-term care insurance assessment, such a development in, e.g., drug efficacy, drug interactions and/or drug side effects can take years (if at all) to be incorporated as a factor in long-term insurance assessment. However, such rapid developments in drug efficacy (or other health related data) can be readily detected by the disclosed system, and acted upon accordingly. Thus, the disclosed technology enables efficient and rapid incorporation of an individual's current status, or a change in individual's status (e.g., levels of activity, deterioration or improvement of health/disability), into the long-term care insurance assessment. As a result, a more accurate assessment of an individual's long-term care needs that are based on relevant and up-to-date information can be produced, which leads to issuance of better long-term care insurance policies.

The components or modules of the disclosed systems can be implemented as hardware, software, or combinations thereof. For example, a hardware implementation can include discrete analog and/or digital circuits that are, for example, integrated as part of a printed circuit board. Alternatively, or additionally, the disclosed components or modules can be implemented as an Application Specific Integrated Circuit (ASIC) and/or as a Field Programmable Gate Array (FPGA) device. Some implementations may additionally or alternatively include a digital signal processor (DSP) that is a specialized microprocessor with an architecture optimized for the operational needs of digital signal processing associated with the disclosed functionalities of this application.

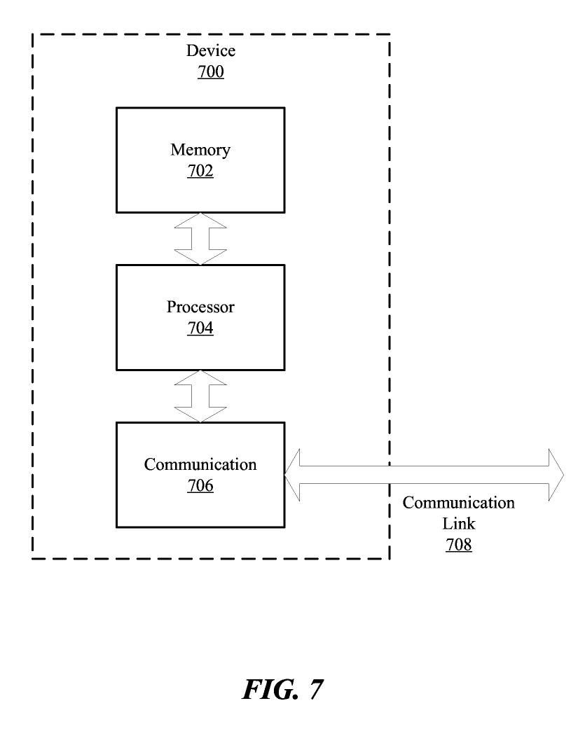

FIG. 7 illustrates a block diagram of a device 700 that can be implemented as part of the disclosed devices and systems. The device 700 comprises at least one processor 704 and/or controller, at least one memory 702 unit that is in communication with the processor 704, and at least one communication unit 706 that enables the exchange of data and information, directly or indirectly, through the communication link 708 with other entities, devices, databases and networks. The communication unit 706 may provide wired and/or wireless communication capabilities in accordance with one or more communication protocols, and therefore it may comprise the proper transmitter/receiver, antennas, circuitry and ports, as well as the encoding/decoding capabilities that may be necessary for proper transmission and/or reception of data and other information. The exemplary device 700 of FIG. 7 may be integrated as part of the devices or components of the disclosed technology, such as the user device, the insurance provider device, the data sources, or the data aggregation and analysis system.

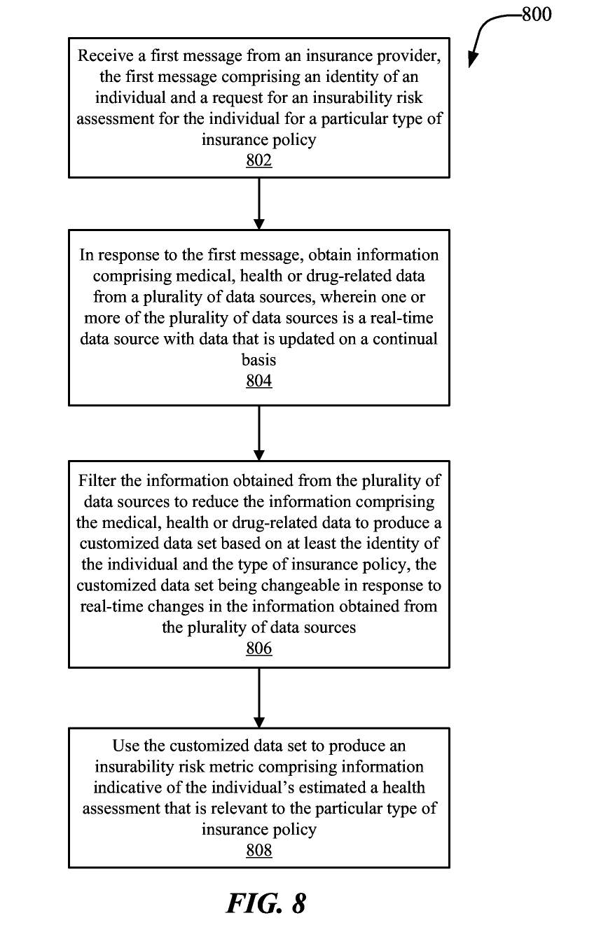

FIG. 8 illustrates a set of exemplary operations 800 that may be carried out to provide an insurance risk metric in accordance with an exemplary embodiment. At 802, a first message is received from an insurance provider. The first message includes an identity of an individual and a request for an insurability risk assessment for the individual for a particular type of insurance policy. At 804, in response to the first message, information comprising medical, health or drug-related data from a plurality of data sources is obtained. One or more of the plurality of data sources is a real-time data source with data that is updated on a continual basis. At 806, the information obtained from the plurality of data sources is filtered to reduce the information comprising the medical, health or drug-related data and to produce a customized data set based on at least the identity of the individual and the type of insurance policy. The customized data set is changeable in response to real-time changes in the information obtained from the plurality of data sources. At 808, the customized data set is used to produce an insurability risk metric comprising information indicative of the individual's estimated a health assessment that is relevant to the particular type of insurance policy.

In one exemplary embodiment, the particular type of insurance policy is one of a health insurance policy, a life insurance policy or a long-term care insurance policy. In another exemplary embodiment, the filtering includes processing the information obtained from the plurality of data sources to remove redundant data and to remove data that is not relevant to the individual or to the type of insurance policy. In one exemplary embodiment, the filtering can produce the customized data set that includes entries that are sorted in a predetermined order. For instance, the predetermined order is based on relevance to the individual or to the type of insurance policy, or based on a time associated with each entry.

As shown earlier in FIG. 5, the plurality of data sources include an insurance claim data source, include a pharmaceutical data source, a behavior data source, a clinical data source, a telematics data source, a law enforcement or government data source, a weather or disaster data source, and a third party data source. The insurance claim data source provides information associated with previously filed insurance claims, cost data describing services that were provided as part of the previously filed insurance claims, and an amount of reimbursement provided for each of the previously filed insurance claims. The pharmaceutical data source provides data associated with therapeutic mechanism of action of one or more drugs, a target behavior in human body, side effects and toxicity of the one or more drugs, and drug trial information obtained as a result of phases 0 through 4 of a discovery process associated with one or more drugs. The behavior data source provides data that describes activities and preferences of the individual and financial data associated with the individual. The clinical data source provides patient data stored in one or more computer-based information system that aggregate patient data for use by physicians, hospitals or as part of a health-information exchange, the clinical data source further providing drug trial information produced as a result of phases 0 through 4 of drug discovery process, and additional data associated with long-term effects, efficacy and issues related to particular drugs. The law enforcement or government data source provides data associated with fraud history, criminal history, residence history or aliases or other names associated with the individual. The weather or disaster data source provides data obtained from agencies that monitor or forecast weather patterns or disasters.

According to one exemplary embodiment, one or more of the plurality of data sources collect at least a part of the medical, health or drug-related data from an online social network. In another embodiment, the request that is received from the insurance provider requires collection and aggregation of specific types of data. In this scenario, in response to the first message, an application specific data source is created to obtain the specific types of data requested in the first message, and to allow generation of the insurability metric based on the specific types of data. In yet another exemplary embodiment, the information obtained from the plurality of data sources includes health related information that is obtained directly from the individual and is produced by a personalized health monitoring device that is capable of obtaining or measuring the individual's health related information and transmitting them to a database.

In one exemplary embodiment, the customized set of data is produced based on an interaction between a first set of data obtained from a first one of the plurality of data sources and at least a second set of data obtained from a second one of the plurality of data sources. In particular, such interaction between the first set of data and the at least second set of data improves the drug trial suitability determination and/or insurability risk assessment for the individual. In another exemplary embodiment, the drug trial suitability and/or insurability risk metric is produced for a predetermined period of time, where the smallest duration of the predetermined period of time is one hour. In still another exemplary embodiment, the drug trial suitability and/or insurability risk metric includes a weighted average insurance risk assessment or of a drug trial suitability values, information identifying a particular statistics-based model was used to produce the drug trial suitability or insurance risk assessment values, and one or more assumptions that were made in producing the drug trial suitability or insurance risk assessment values based on the particular statistics-based model. In another exemplary embodiment, the drug trial suitability and/or insurability risk metric is produced based on a information obtained from a law enforcement or government data source that allows a determination of a true identity of the individual based on aliases or former names of the individual, and wherein the filtering comprises producing the customized data set that is based on the true identity of the individual.

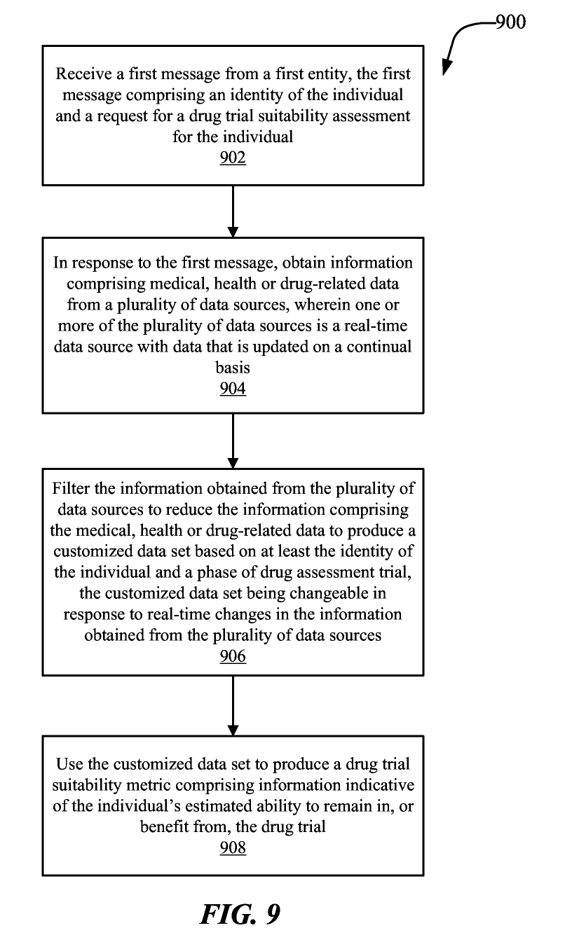

As described herein, the disclosed technology can be used to facilitated drug discovery trials and to assess the suitability of individuals to participate and benefit from a drug trial or consumption of a particular drug. In particular, FIG. 9 illustrates a set of exemplary operations 900 that can be used to assess an individual's suitability to participate in, or benefit from, a drug assessment trial. At 902, a first message is received from a first entity. The first message includes an identity of the individual and a request for a drug trial suitability assessment for the individual. At 904, in response to the first message, information comprising medical, health or drug-related data is obtained from a plurality of data source. One or more of the plurality of data sources is a real-time data source with data that is updated on a continual basis.

At 906, the information obtained from the plurality of data sources is filtered to reduce the information comprising the medical, health or drug-related data, and to produce a customized data set based on at least the identity of the individual and a phase of drug trial. The customized data set is changeable in response to real-time changes in the information obtained from the plurality of data sources. At 908, the customized data set is used to produce a drug trial suitability metric comprising information indicative of the individual's estimated ability to remain in, or benefit from, the drug trial.

In one exemplary embodiment, the customized set of data includes information regarding current list of medications, and a level of fitness of the individual as determined, for example, from data produced by a personalized health monitoring device that is capable of obtaining or measuring the individual's health related information and transmitting them to a database. In another exemplary embodiment, the customized set of data includes information indicative of the individual's response to other drugs or treatments. In still another exemplary embodiment, the customized set of data includes information associated with long-term or short-term efficacy and side effects of the drug. In yet another exemplary embodiment, the customized set of data includes information regarding current list of medications, and a level of fitness of the individual as determined from data produced by a personalized health monitoring device that is capable of obtaining or measuring the individual's health related information and transmitting the individual's health related information to a database.

Referring back to FIG. 5, many of the same components can be used to produce the desired risk assessment metrics for drug trial and discovery purposes. For example, in one implementation, the decision engine 532 includes an algorithm that implements a predetermined model such as statistics-based model. The decision engine 532 can use several parameters that are produced by the filter engine 526 that are considered important in estimating the individual's ability to remain in, or benefit from, the drug trial. Such parameters can include age and body mass index (BMI) of the person, family history of diseases, level of daily exercise, the individual's response to other (or perhaps similar) drugs or treatments, the current medications that the individual is taking, current health issues that the individual is experiencing, and the like. In producing the suitability metric for drug trail purposes, the model can assign particular weights to each of these parameters in producing a weighted average suitability index (e.g., in the range 1 to 100) that is indicative of the individual's estimated ability to remain in, or benefit from, the drug trial in the next month, next six months or next year. In some embodiments, the metric also includes information as to the particular statistics-based model that was used to produce the assessments, and any assumptions that may have been made in producing the assessments. In one embodiment, the metric can include several sets of assessment data (e.g., based on different models, based on different assumptions, for different types of drugs that are being considered, etc.).

Referring back to FIGS. 4 and 5, one aspect of the disclosed embodiments is a system for determination of suitability of an individual for participation in a drug assessment trial. Such a system includes a data aggregation and analysis component that can be implemented at least partially using electronic circuits. The data aggregation and analysis component includes a front end, an identification engine, a customization engine, a filter engine, a decision engine and a non-transitory computer readable storage. Such a system also includes a plurality of data sources coupled to at least the data aggregation and analysis component. Whereas, in FIG. 4, the insurance provide device initiates a request, in embodiments that produce drug trial related assessments a requesting device (e.g., one that is owned by a pharmaceutical company, a clinical research organization (CRO), or another entity) initiates a request for drug trail assessment. As such, in the diagram of FIG. 5, the front end is coupled to at least a communication link and includes an interface to receive data or information from one or more of: a client device, a requesting device, or the plurality of data sources. The identification engine is coupled to at least the front end to receive an identity of an individual and to authenticate the identity, and the customization engine is coupled to the front end to receive information provided by the requesting device indicative of a request for a drug trial suitability assessment for the individual. The filter engine is coupled to at least the plurality of data sources an the non-transitory computer readable storage to obtain information comprising medical, health or drug-related data from the plurality of data sources including at least one real-time data source with data that is updated on a continual basis, and the filter engine filters the information obtained from the plurality of data sources to reduce the information comprising the medical, health or drug-related data to produce a customized data set based on at least the identity of the individual and a phase of drug assessment trial. Such customized data set is changeable in response to real-time changes in the information obtained from the plurality of data sources. The decision engine is coupled to at least the filter engine to use the customized data set to produce a drug trial suitability metric comprising information indicative of the individual's estimated ability to remain in, or benefit from, the drug trial.

Various embodiments described herein are described in the general context of methods or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), Blu-ray Discs, etc. Therefore, the computer-readable media described in the present application include non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

What is claimed is:

1. A system for determining suitability of an individual for participation in a drug assessment trial, the system comprising:
a data aggregation and analysis component implemented at least partially using electronic circuits, and comprising a front end, an identification engine, a customization engine, a filter engine, a decision engine and a non-transitory computer readable storage; and
a plurality of data sources coupled to at least the data aggregation and analysis component, wherein
the front end is coupled to at least a communication link and includes an interface to receive data or information from one or more of: a client device, a requesting device, or the plurality of data sources,
the identification engine is coupled to at least the front end to receive an identity of an individual and to authenticate the identity,
the customization engine is coupled to the front end to receive information provided by the requesting device indicative of a request for a drug trial suitability assessment for the individual,
the filter engine is coupled to at least the plurality of data sources and the non-transitory computer readable storage to obtain information comprising medical, health or drug-related data from the plurality of data sources including at least one real-time data source with data that is updated on a continual basis, the filter engine to filter the information obtained from the plurality of data sources to reduce the information comprising the medical, health or drug-related data to produce a customized data set based on at least the identity of the individual and a phase of drug assessment trial, the customized data set being changeable in response to real-time changes in the information obtained from the plurality of data sources, and
the decision engine is coupled to at least the filter engine to use the customized data set to produce a drug trial suitability metric comprising information indicative of the individual's estimated ability to remain in, or benefit from, the drug trial.

2. The system of claim 1, wherein the drug trial suitability metric includes a weighted average suitability index, information identifying a particular statistics-based model that was used to produce the suitability index, and one or more assumptions that were made in producing the suitability index values based on the particular statistics-based model.

3. The system of claim 1, wherein the customized set of data includes information regarding current list of medications, and a level of fitness of the individual as determined from data produced by a personalized health monitoring device that is capable of obtaining or measuring the individual's health related information and transmitting the individual's health related information to a database.

4. The system of claim 1, wherein the customized set of data includes information indicative of the individual's response to other drugs or treatments.

5. The system of claim 1, wherein the customized set of data includes information associated with long-term or short-term efficacy and side effects of the drug.

6. The system of claim 1, wherein the plurality of data sources include information related to one or more of: a health-related outbreak, a natural disaster or a personalized health profile.

7. The system of claim 1, wherein the plurality of data sources include a clinical data source, an insurance claim data source, and a pharmaceutical research and development data source.

8. The system of claim 7, wherein, the clinical data source includes:
information obtained from a basic electronic medical record or a health-information exchange,
a result of phases 0 through 4 of a drug discovery process, and
data associated with long-term effects, efficacy or issues related to particular drugs.

9. A method for determination of suitability of an individual for participation in a drug assessment trial, comprising:
receiving a first message from a first entity, the first message comprising an identity of the individual and a request for a drug trial suitability assessment for the individual;
in response to the first message, obtaining information comprising medical, health or drug-related data from a plurality of data sources, wherein one or more of the plurality of data sources is a real-time data source with data that is updated on a continual basis;
filtering the information obtained from the plurality of data sources to reduce the information comprising the medical, health or drug-related data to produce a customized data set based on at least the identity of the individual and a phase of drug assessment trial, the customized data set being changeable in response to real-time changes in the information obtained from the plurality of data sources; and
using the customized data set to produce a drug trial suitability metric comprising information indicative of the individual's estimated ability to remain in, or benefit from, the drug assessment trial.

10. The method of claim 9, wherein the customized set of data includes information regarding current list of medications, and a level of fitness of the individual as determined from data produced by a personalized health monitoring device that is capable of obtaining or measuring the individual's health related information and transmitting the individual's health related information to a database.

11. The method of claim 9, wherein the customized set of data includes information indicative of the individual's response to other drugs or treatments.

12. The method of claim 9, wherein the customized set of data includes information associated with long-term or short-term efficacy and side effects of the drug.

13. The method of claim 9, wherein the drug trial suitability metric includes a weighted average suitability index, information identifying a particular statistics-based model that was used to produce the suitability index, and one or more assumptions that were made in producing the suitability index values based on the particular statistics-based model.

14. The method of claim 9, wherein the plurality of data sources include information related to one or more of: a health-related outbreak, a natural disaster or a personalized health profile.

15. The method of claim 9, wherein the plurality of data sources include a clinical data source, an insurance claim data source, and a pharmaceutical research and development data source.

16. The method of claim 15, wherein, the clinical data source includes:
information obtained from a basic electronic medical record or a health-information exchange,
a result of phases 0 through 4 of a drug discovery process, and
data associated with long-term effects, efficacy or issues related to particular drugs.

17. A computer program product, embodied on one or more computer readable media, comprising:
program code for receiving a first message from a first entity, the first message comprising an identity of the individual and a request for a drug trial suitability assessment for the individual;
program code for, in response to the first message, obtaining information comprising medical, health or drug-related data from a plurality of data sources, wherein one or more of the plurality of data sources is a real-time data source with data that is updated on a continual basis;
program code for filtering the information obtained from the plurality of data sources to reduce the information comprising the medical, health or drug-related data to produce a customized data set based on at least the identity of the individual and a phase of drug assessment trial, the customized data set being changeable in response to real-time changes in the information obtained from the plurality of data sources; and
program code for using the customized data set to produce a drug trial suitability metric comprising information indicative of the individual's estimated ability to remain in, or benefit from, the drug trial.

18. The computer program product of claim 17, wherein the customized set of data includes information regarding current list of medications, and a level of fitness of the individual as determined from data produced by a personalized health monitoring device that is capable of obtaining or measuring the individual's health related information and transmitting the individual's health related information to a database.

19. The computer program product of claim 17, wherein the customized set of data includes information indicative of the individual's response to other drugs or treatments.

20. The computer program product of claim 17, wherein the customized set of data includes information associated with long-term or short-term efficacy and side effects of the drug.

21. The computer program product of claim 17, wherein the drug trial suitability metric includes a weighted average suitability index, information identifying a particular statistics-based model that was used to produce the suitability index, and one or more assumptions that were made in producing the suitability index values based on the particular statistics-based model.

22. The computer program product of claim 17, wherein the plurality of data sources include information related to one or more of: a health-related outbreak, a natural disaster or a personalized health profile.

23. The computer program product of claim 17, wherein the plurality of data sources include a clinical data source, an insurance claim data source, and a pharmaceutical research and development data source.

24. The computer program product of claim 23, wherein, the clinical data source includes:
information obtained from a basic electronic medical record or a health-information exchange,
a result of phases 0 through 4 of a drug discovery process, and
data associated with long-term effects, efficacy or issues related to particular drugs.
